(19) United States
(12) Patent Application Publication
(10) Pub. No.: US 2024/0321467 A1
(43) Pub. Date: Sep. 26, 2024

(54) SYSTEM AND METHOD FOR DEVELOPING, MAINTAINING, AND IMPLEMENTING A PRODUCT FORMULARY FOR MEDICAL PRODUCT ITEMS IN A DIGITAL MARKETPLACE

(71) Applicant: Libertas Implant Solutions, LLC, Arlington, VA (US)

(72) Inventors: Joseph T. BUTZ, Potomac, MD (US); Nathaniel GIVENS, Ashland, VA (US); Kenneth Thomas ARMSTRONG, Perrysburg, OH (US)

(73) Assignee: Libertas Implant Solutions, LLC, Arlington, VA (US)

(21) Appl. No.: 18/610,326

(22) Filed: Mar. 20, 2024

Related U.S. Application Data
(60) Provisional application No. 63/453,291, filed on Mar. 20, 2023.

(51) Int. Cl. G16H 70/20

(52) U.S. Cl. CPC G16H 70/20

(57) ABSTRACT

The present disclosure provides a description of systems and methods for creation of a product formulary for medical product items. A clinical usage hierarchy is created for a practice area that includes multiple organizational levels and substitutable group categories. Data for known, categorizable products is entered into the categories of the clinical usage hierarchy and used to train a machine learning model, which is then deployed on a universal item database to populate the clinical usage hierarchy with all possible substitute items according to available categorization information in the database. This resulting clinical usage hierarchy has populated categories of items used in procedures in the practice area, where each item in a category is substitutable for any other item in that category, and is used to generate a product formulary utilized by physician or healthcare providers when selecting items for a future medical procedure in the practice area.

### FIELD

The present disclosure relates to developing, maintaining, and implementing a product formulary, specifically the use of artificial intelligence and a digital marketplace to facilitate an efficient process for obtaining medical product items (e.g., physician preference items) in compliance with all applicable regulations.

### BACKGROUND

The medical industry is a massive and intricate industry where the needs and requirements of physician or healthcare providers are as varied and complicated as the patients they serve. There are thousands of different medical product items available for use by physician or healthcare providers to treat patients coming from a vast number of different manufacturers. It can be exceedingly difficult for a physician or healthcare providers to keep apprised of the various manufacturers, their products, and identify potential alternatives to known products. As such, physician or healthcare providers often rely on referrals from colleagues or direct sales from manufacturer representatives and tend to stick with known, familiar products. This can lead to significantly increased costs for a physician or healthcare provider while also preventing the physician or healthcare provider from identifying new, and sometimes more effective, tools and products.

As technology improves, some platforms have been created to simplify the distribution of medical product items. These platforms often resemble e-commerce retailers, but where the product formulary is limited to medical products. These platforms can often enable a physician or healthcare providers to browse through product listings, but do not evaluate product items or provide any information to aid the physician or healthcare providers in selecting products, particularly when it comes to more specific clinical needs, medical product item technology offerings, device pricing and clinical outcomes data, etc. As such, physician or healthcare providers tend to use familiar products or make purchases with specific recommendations from colleagues, resulting in such platforms not improving clinical decision making, optimal technology utilization, nor optimal pricing based on device offerings.

When it comes to medications, drug formularies are used to aid physicians or healthcare providers in the selection, prescription, and use of medication. A drug formulary, also known as a preferred drug list, is a continually updated list of medications that is curated based on available medical knowledge and research, physician and pharmacist input, clinical and patient needs, and evaluation of clinical and medical literature. A physician or healthcare providers treating a specific symptom or ailment can consult the drug formulary to identify a suitable medication and, if that medication is unavailable or cost prohibitive in the situation, other, alternative medications.

However, formularies are limited to medications. In the similar scenario where a physician or healthcare providers may need a medical product item, but where such an medical product item is sold out, cost prohibitive, or otherwise unavailable, the physician is left with little ability to locate an alternative medical product item, which is particularly challenging if the medical product item is needed for a medical procedure, proper patient care or some other care delivery or operational. Consequently, a need exists for a formulary for medical products that can serve the needs of physician or healthcare providers to obtain products that can ensure proper clinical care as well as compliance with all applicable rules and regulations.

### SUMMARY

The present disclosure provides a description of systems and methods for creation of a product formulary for medical product items (e.g., physician preference items). A clinical usage hierarchy is created for a practice area that includes multiple organizational levels and a mix of pass-through categories and substitutable group categories. Data for known, categorizable products is entered into the clinical usage hierarchy in the appropriate categories and used to train a machine learning model. The machine learning model is then deployed on a universal item database to populate the clinical usage hierarchy with all possible substitute items according to available categorization information in the universal item database. This results in a clinical usage hierarchy that is broken into categories for items used in procedures in the practice area, where each item in a category is substitutable for any other item in the same category. A product formulary is generated using the clinical usage hierarchy that can be utilized by physician or healthcare providers when selecting items for a future medical procedure in the practice area. When the ability to substitute items within a category is attenuated by a very wide range of quality or relevant features, the product formulary can be further enhanced by dividing the category into two or more grades. This begins with the designation of clinically relevant attributes for each item in the substitutable group categories. A clinically relevant attribute hierarchy can be defined for each substitutable group category and another machine learning model trained on items in each category whose clinically relevant attributes are known and deployed on the remaining items in that category in order to populate the clinically relevant attribute hierarchy with clinically relevant attributes for that item. The enhanced product formulary can further aid physician or healthcare providers in the selection of preference items due to the clinically relevant attributes, which can also be used to grade the items in a substitutable group category into separate tiers for further assistance in selection by physician or healthcare providers, where such grading can be performed by another trained machine learning algorithm. The product formulary can provide significant advantages over existing systems by (1) making all possible substitutable items quickly identifiable and ready for evaluation by a physician or healthcare providers, (2) ensuring that the product formulary is quickly and accurately created and kept up-to-date with minimal-to-no user interaction, (3) providing medical device comparisons for optimal clinical and pricing utilizations, and (4) ensuring compliance with regulatory and statutory requirements. In some cases, a product formulary can be used as part of a digital marketplace to facilitate the procurement of medical product items (physician preference items) in addition to selection of items for a procedure.

A method for creation of a product formulary for medical product items includes: determining, by a processor of a processing system, a clinical usage hierarchy for a practice area, the clinical usage hierarchy including at least two organizational levels, where the first organizational level includes one or more organizational pass-through categories and where the second organizational level includes at least one substitutable group category; receiving, by a receiver of the processing system, one or more item entries for each substitutable group category included in the clinical usage hierarchy, wherein each of the one or more item entries includes at least item categorization information; training, by the processor of the processing system, a first machine learning model using the one or more items for each substitutable group category in the clinical usage hierarchy; deploying, by the processing system, the trained first machine learning model on a universal item database populated with a plurality of item data entries to identify a subset of item data entries of the plurality of item data entries for each substitutable group category included in the clinical usage hierarchy, where each item data entry of the plurality of item data entries includes at least one or more product categories, and the machine learning model identifies the subset of item data entries for each substitutable group category based on at least the one or more product categories included in each item data entry of the subset of item data entries and the item categorization information in the one or more item data entries included in the respective substitutable group category; storing, in a memory of the processing system, the clinical usage hierarchy, each substitutable group category in the clinical usage hierarchy, and, in each substitutable group category, the one or more item entries and the identified subset of item data entries for the respective substitutable group category; and generating, by the processor of the processing system, a product formulary for the practice area based on at least the stored clinical usage hierarchy, wherein an item associated with an item entry item data entry in each substitutable group category in the clinical usage hierarchy is substitutable with another item associated with an item entry item data entry in the same substitutable group category for a procedure associated with the practice area.

A system for creation of a product formulary for medical product items includes one or more processors, and a non-transitory computer readable medium storing instructions when executed by the one or more processors cause the one or more processors to: determine a clinical usage hierarchy for a practice area, the clinical usage hierarchy including at least two organizational levels, where the first organizational level includes one or more organizational pass-through categories and where the second organizational level includes at least one substitutable group category; receive one or more item entries for each substitutable group category included in the clinical usage hierarchy, wherein each of the one or more item entries includes at least item categorization information; train a first machine learning model using the one or more items for each substitutable group category in the clinical usage hierarchy; deploy the trained first machine learning model on a universal item database populated with a plurality of item data entries to identify a subset of item data entries of the plurality of item data entries for each substitutable group category included in the clinical usage hierarchy, where each item data entry of the plurality of item data entries includes at least one or more product categories, and the machine learning model identifies the subset of item data entries for each substitutable group category based on at least the one or more product categories included in each item data entry of the subset of item data entries and the item categorization information in the one or more item data entries included in the respective substitutable group category; store, in a memory, the clinical usage hierarchy, each substitutable group category in the clinical usage hierarchy, and, in each substitutable group category, the one or more item entries and the identified subset of item data entries for the respective substitutable group category; and generate a product formulary for the practice area based on at least the stored clinical usage hierarchy, wherein an item associated with an item entry item data entry in each substitutable group category in the clinical usage hierarchy is substitutable with another item associated with an item entry item data entry in the same substitutable group category for a procedure associated with the practice area.

### BRIEF DESCRIPTION OF THE DRAWING FIGURES

The scope of the present disclosure is best understood from the following detailed description of exemplary embodiments when read in conjunction with the accompanying drawings. Included in the drawings are the following figures:

FIG. 1 is a diagram illustrating a generic clinical usage hierarchy for the creation of a product formulary in accordance with exemplary embodiments.

FIG. 2 is a diagram illustrating clinical usage hierarchy for a cardiac rhythm management practice area for a product formulary in accordance with exemplary embodiments.

FIG. 3 is a diagram illustrating the population of items in graded substitutable group categories of a clinical usage hierarchy using a universal item database and machine learning in accordance with exemplary embodiments.

FIG. 4 is a diagram illustrating the designation of clinically relevant attributes for a clinically relevant attribute hierarchy for items in a graded substitutable group category for training a machine learning model in accordance with exemplary embodiments.

FIG. 5 is a diagram illustrating the grading of medical product items into tiers in a graded substitutable group category using a machine learning model in accordance with exemplary embodiments.

FIG. 6 is a diagram illustrating a clinical usage hierarchy that includes systems of items from multiple substitutable group categories in accordance with exemplary embodiments.

FIG. 7 is a flow diagram illustrating a process for entering procedures and selecting and purchasing medical product items for a procedure using a product formulary in a digital marketplace in accordance with exemplary embodiments.

FIG. 8 is a flow diagram illustrating a process for matching an invoice with a completed procedure report in a digital marketplace in accordance with exemplary embodiments.

FIG. 9 is a diagram illustrating a high-level system architecture for developing, maintaining, and implementing a product formulary in accordance with exemplary embodiments.

FIG. 10 is a flow diagram illustrating an exemplary method for creation of a product formulary for medical product items in accordance with exemplary embodiments.

FIG. 11 is a block diagram illustrating a computer system architecture in accordance with exemplary embodiments.

Further areas of applicability of the present disclosure will become apparent from the detailed description provided hereinafter. It should be understood that the detailed description of exemplary embodiments is intended for illustration purposes only and are, therefore, not intended to necessarily limit the scope of the disclosure.

### DETAILED DESCRIPTION

### Creation of a Clinical Usage Hierarchy

The methods and systems discussed herein are directed toward the development, maintenance, and implementation of a product formulary for medical product items (e.g., physician preference items). A product formulary is defined for a particular practice area and includes all of the medical product items within that practice area. Items in a product formulary are organized into a clinical usage hierarchy to create groups of substitutable items within and among manufacturers. In this context, two items are substitutable if, for a typical procedure, either one could feasibly be used. Substitutability in this context does not imply that the items are identical in all respects, nor that they could be substituted for each other in every possible instance, nor that they would yield exactly the same outcomes in all cases. Instead, substitutability only implies that either of two items could be substituted for the other to accomplish the main objective of a typical procedure. In economic terms, items need not be perfect substitutes to be considered substitutable. Additional item differentiation (“grading”, see below) is necessary in some cases to yield sufficient substitutability within some categories to enable genuine price competition.

In addition to a clinical usage hierarchy, a product formulary includes a list of clinically relevant attributes that are populated for all items on the basis of which the items within a substitutable group can be objectively divided into distinct price levels using the clinically relevant attributes. This can provide for a clinically relevant basis for distinguishing between new or enhanced technology and older or more basic technology within a substitutable group, which can assist in medical product item selection and provide for more cost-effective treatment.

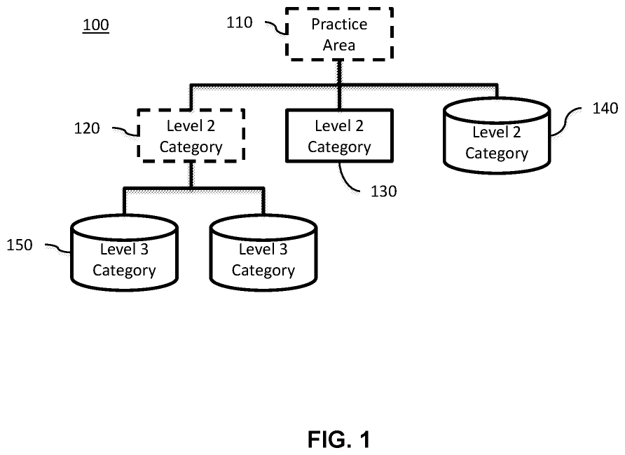

FIG. 1 illustrates an example of a clinical usage hierarchy 100 used to organize items within a practice area into a hierarchy of categorizations and to identify groups of substitutable items that will be priced and graded. The items within a substitutable group can be further broken down into clusters defined by their clinically relevant attributes and then graded (e.g., low, medium, high, etc.) where the grades can offer either objectively superior or contingently superior benefits. In general, for a given procedure any of the items in the substitutable group could feasibly be chosen.

The top level of the clinical usage hierarchy 100 is the practice area 110. The practice area 110 is the area to which the items pertain. The practice area 110 can be a specific procedure (e.g., meniscus repair), an area of medicine (e.g., orthopedics), or other area of organization that is suitable for performing the functions discussed herein. Underneath the clinical usage hierarchy 100 can be one of three different types of categories. The first type is an organizational pass-through category 120, which is a category that contains no items within the category itself. The second type is a non-graded substitutable group category 130, which can contain a group of substitutable items from which a physician or healthcare provider can reasonably choose any particular item for a particular procedure, but where the items are not individually graded or priced. The third type of category is a graded substitutable group category 140, which can include items from which a physician or healthcare provider can reasonably chose any particular item for a particular procedure where the items are individually graded and priced.

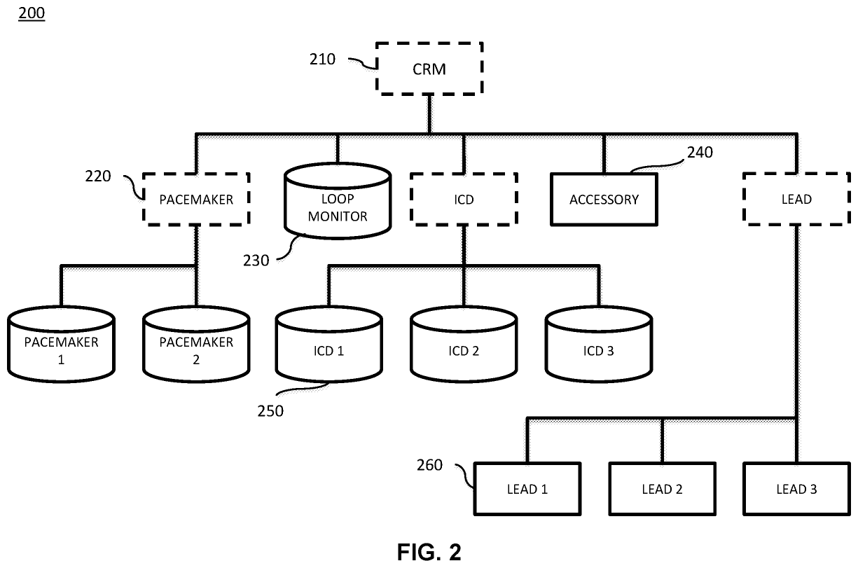

The example illustrated in FIG. 1 is for a generic clinical usage hierarchy 100 to illustrate the types of categories and possible organization of the clinical usage hierarchy 100. In the generic example, there are top-level categories of each type, and where the organizational pass-through category 120 includes two subcategories 140, both of which are graded substitutable group categories. A detailed example of a clinical usage hierarchy 100 for a specific practice area 110 is illustrated in FIG. 2. FIG. 2 illustrates a clinical usage hierarchy 200 for a practice area 210 for cardiac rhythm management (CRM) for which the clinical usage hierarchy 200 is developed to create a CRM-specific product formulary. In the example, the top level of the clinical usage hierarchy 200 can include organizational pass-through categories 220 for pacemakers, implantable cardioverter-defibrillators (ICDs), and leads, a graded substitutable group category 230 for loop monitors, and a non-graded substitutable group category 240 for accessories. In the CRM practice area 210, each of the substitutable group categories 220 also includes sub-groups at a lower level. The pacemaker and ICD groups include graded substitutable group sub-categories 250, while the lead group includes non-graded substitutable group sub-categories 260.

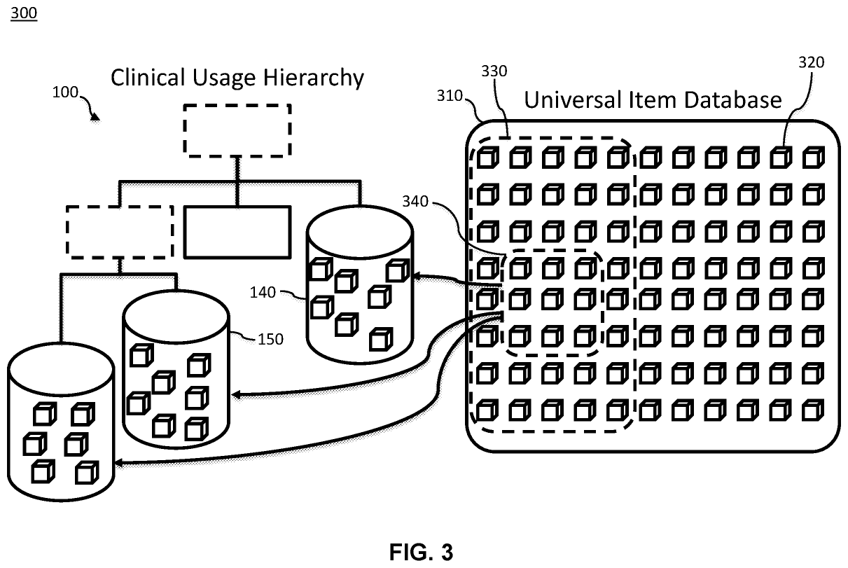

After a clinical usage hierarchy 100 for a practice area 110 is completed, the categories are filled with items. Items can be identified for inclusion in the various categories of the clinical usage hierarchy 100 from a database of items. FIG. 3 includes a system 300 that illustrates the population of the categories in a clinical usage hierarchy 100 with items. The system 300 can include a universal item database 310. The universal item database 310 can be a database that is configured to store detailed information on a plurality of different medical products. For example, the Food and Drug Administration (FDA) operates a Global Unique Device Identification Database (GUDID) that provides a list of all medical devices approved in the United States and gives categorization information on each item in the list. Examples discussed herein will utilize the FDA's GUDID as the universal item database 310, but it will be apparent to persons having skill in the relevant art that any other item database that provides categorization information on items stored therein can be utilized with the methods and systems discussed herein.

In the system 300, the universal item database can store information for a plurality of different items 320. Information included for each item can include at least a category identifier, as well as any other information associated with the product, which can depend on the type of product, such as serial numbers, lot numbers, manufacturer name, manufacture date, authorization information, etc. For instance, the FDA GUDID utilizes two different sources of item categorization: FDA product codes, which are 3-letter codes provided by the FDA, and global medical device nomenclature (GMDN) information, which is defined in the International Organization of Standardization's ISO 15225 standard. In many instances, there are significant overlaps between the FDA's product codes and the GMDN categories, but there are also many items 320 where the categories can differ. As such, it can be practically impossible to directly populate categories in a clinical usage hierarchy 100 for any practice area 110 from the universal item database 310 using the categorization information alone.

In order to identify all of the items 320 that are related to a specific practice area 110, the system 300 can utilize a combination of artificial intelligence and machine learning. A machining learning model is trained using sample data for known products in non-graded substitutable group categories 130 and graded substitutable group categories 140 for a known practice area 110. The known products in established groups in the clinical usage hierarchy 100 can be mapped to corresponding fields in the universal item database 310 for the product as an item 320 in the universal item database 310. The FDA product code and GMDN information, or other categorization information, as applicable, can be identified and included in a data table that includes each of the known products. The data table with available product information, categorization in the clinical usage hierarchy 100, and categorization information from the universal item database 310, can serve as the training data for the machine learning model. The type of machine learning model used can vary depending on the exact product attributes that are available, the categories in the clinical usage hierarchy 100, the specific practice area 110, etc. In some cases, baseline predictive values in the machine learning model will be stored for later use in maintenance of the product formulary, as discussed below.

After the machine learning model is trained, an artificial intelligence engine or other suitable processing system can deploy the machine learning model on the universal item database 310 to identify all items 320 that should be included in one of the categories in the clinical usage hierarchy 100 on which the machine learning model was trained. In the example illustrated in FIG. 2, the machine learning model can be trained on known product data for the CRM practice area 210 to identify all of the other items 320 in the FDA's GUDID database that should be included in the non-graded substitutable group categories 240 and 260 and graded substitutable group categories 230 and 250 in the clinical usage hierarchy 200 for the CRM practice area 210. The machine learning model can identify a set 330 of items 320 in the universal item database 310 whose categorization and other information are determined to be suitable for use in the specific practice area 110, and further identify a subset 340 of items that should be included as graded items. The system 300 can then populate the non-graded substitutable group categories 130 in the clinical usage hierarchy 100 with the identified non-graded items 310 from the set 330 and populate the graded substitutable group categories 140 and 150 of the clinical usage hierarchy 100 with the identified graded items 310 from the subset 340.

By using a trained machine learning model and a universal item database 310, the clinical usage hierarchy 100 can be kept up-to-date and accurate any time changes or additions to the universal item database 310 occur. The machine learning model can be further trained using the updated clinical usage hierarchy 100 after items have been added, such as following successful usage, feedback, user input, etc. For instance, a user, such as a physician specializing in the specific practice area 110, can make adjustments, confirmations, etc. to categorizations made by the machine learning model, where such confirmed data can be included in the training data for further training of the machine learning model, such as to provide greater accuracy for future deployments on the universal item database 310, such as when item information changes or new items 320 are added to the universal item database 310.

### Designation and Use of Clinically Relevant Attributes

After a clinical usage hierarchy 100 has been created and populated with items 320 from a universal item database 310, items that are placed in the graded substitutable group categories 140 need to be graded. In order to grade substitutable items, clinically relevant attributes need to be identified for each of the items in the category.

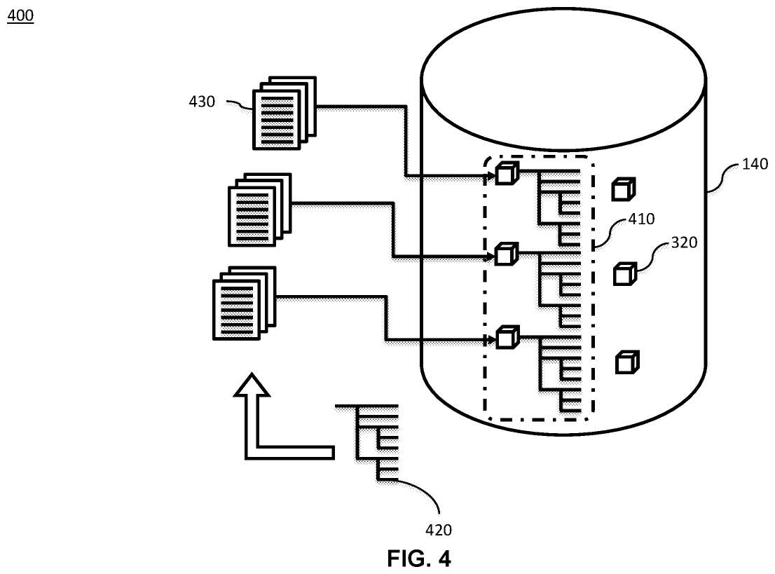

FIG. 4 illustrates a system 400 for the designation of clinically relevant attributes for use in grading items 320 in graded substitutable group categories 140. In order to grade all of the items 320 in the graded substitutable group category 140, a clinically relevant attribute hierarchy 420 needs to be identified for the category 140. The clinically relevant attribute hierarchy 420 is a nested list of attributes that are clinically relevant for differentiating items in the specific graded substitutable group category 140. The attributes can vary depending on the category, its level, the practice area 110, etc. for the specific clinical usage hierarchy 100. Attributes that are included in a clinically relevant attribute hierarchy 420 can include, for example, battery life, operating temperature, material, power usage, weight, dimensions, tension strength, etc. The clinically relevant attribute hierarchy 420 can be represented using any suitable method for presentation of a hierarchy of data, such as using JavaScript Object Notation (JSON), YAML, etc. Each specific graded substitutable group category 140 in a clinical usage hierarchy 100 can have a distinct clinically relevant attribute hierarchy 420. For instance, in the example clinical usage hierarchy 200 for the CRM practice area 210 in FIG. 2, the clinically relevant attribute hierarchy 420 for items 320 in the pacemaker substitutable group categories 250 will be different from the clinically relevant attribute hierarchy 420 for items 320 in the ICD substitutable group categories 250.

After the clinically relevant attribute hierarchy 420 has been identified for the graded substitutable group category 140, a subset 410 of items 320 in the graded substitutable group category 140 for which detailed information can be obtained is identified. For instance, each item 320 in the subset 410 can include items for which manufacturer specifications or other data sheets, illustrated in FIG. 4 as specifications 430, can be obtained. For each of the items 320 in the subset 410, data can be parsed from the specifications 430 in order to populate the attributes in the clinically relevant attribute hierarchy 420. FIG. 4 illustrates that each of the items 320 in the subset 410 includes a populated list of attributes for the identified clinically relevant attribute hierarchy 420, while the clinically relevant attributes for the items 320 not included in the subset 410 are still in need of identification. In some cases, the parsing of clinically relevant attributes from specifications 430 can be an automated process using a specialized program, including a trained machine learning model, or can be done manually by a suitable user (e.g., a physician or other subject matter expert familiar with the practice area 110 or specific substitutable group category 140).

After the clinically relevant attributes have been identified and populated for each of the items 320 in the subset 410, a machine learning model is trained to extract values for the clinically relevant attribute hierarchy 420 for each of the items 320 in the subset 410 as a sample population for training. Once the machine learning model has been trained, the machine learning model can be deployed on the other items 320 of the graded substitutable group category 140 that are not included in the subset 410 to populate the clinically relevant attributes in the applicable clinically relevant attribute hierarchy 420 for the other items 320. The process can be repeated for each of the graded substitutable group categories 140 and 150 in the clinical usage hierarchy 100 for the practice area 110. In some cases, the same process can be used to designate clinically relevant attributes for all items 320 in non-graded substitutable group categories 130 as well.

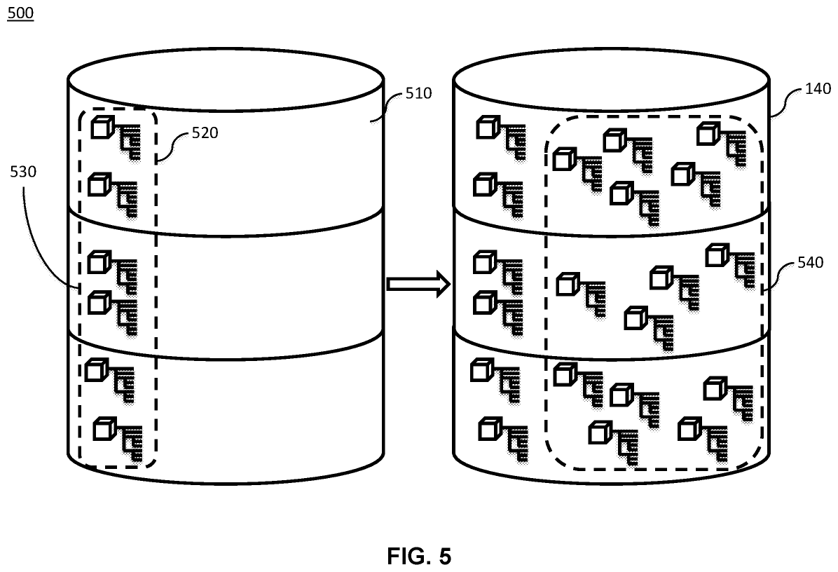

Once the clinically relevant attributes have been designated for each of the items 320 in a graded substitutable group category 140, the items 320 can be graded. FIG. 5 illustrates a process 500 for the grading of items 320 in a graded substitutable group category 140. First, the graded substitutable group category 140 can be broken down into two or more tiers 510. Each tier 510 can represent a division of the items 320 in the graded substitutable group category 140 that can be a useful separation of the items 320 for selection by a physician or other healthcare professional. The tiers 510 can be specific to one or more clinically relevant attributes (e.g., tiers based on cost, product material, etc.), or can be based on several clinically relevant attributes through the use of weighting, algorithms, physician feedback, or other suitable data. In an example, the tiers 510 for a substitutable group category 140 can include standard, plus, and premium.

In order to grade the items 320, a subset of gradable items 520 is first identified. The subset of gradable items 520 can include all of the items 320 in the substitutable group category 140 that can be directly graded using known or otherwise available data. For instance, a user (e.g., a physician or other subject matter expert for the practice area 110) can manually sort the items 320 included in the subset of gradable items 520 into the identified tiers 510. In another example, a processing system can automatically sort the items 320 included in the subset of gradable items 520 into the identified tiers 510 based on accessible data, such as physician feedback, reviews, usage statistics, etc.

Once the items 320 in the subset of gradable items 520 have been sorted into the tiers 510, a machine learning model is trained on the subset of gradable items 520. The machine learning model uses the subset of gradable items 520, the specific sorting of each of the items 320 into the specific tiers 510, and the clinically relevant attribute hierarchy 420 and attributes thereof for the items 320, as a sample population for training. The machine learning model can then be deployed on the rest of the items 320 in the graded substitutable group category 140, represented as remaining subset 540 in FIG. 5. The machine learning model grades each of the items 320 in the remaining subset 540 and sorts the items 320 into the tiers 510. The same process 500 can be repeated with each of the graded substitutable group categories 140 and 150 in the clinical usage hierarchy 110 to ensure that every item 320 in all of the graded categories is successfully graded.

The completed clinical usage hierarchy 100 with the designated clinically relevant attributes and finished grading is considered a product formulary that can be used by a physician to select products for a procedure or other use in the related practice area 110. The use of graded and non-graded grouping of substitutable items ensures that alternative, yet just as suitable and effective, items can be easily identified and, in cases where the items are graded, items of a desired tier can be quickly differentiated and selected. The result is a significantly easier, more effective process for the selection of medical product items over existing platforms and systems. As discussed below, a digital marketplace can be used to enhance a physician or healthcare professional's interaction with the product formulary.

In some cases, the machine learning model used for grading the items 320 in the graded substitutable group category 140 can be the same machine learning model used in the system 300 of FIG. 3 and/or the system 400 of FIG. 4. In other cases, the machine learning model may be unique to the process 500 illustrated in FIG. 5. In some instances, the machine learning model used in the process 500 can be an unsupervised or semi-supervised machine learning model due, particularly in instances where the number of items 320 in a graded substitutable group category 140 are small and/or the number of clinically relevant attributes in the clinically relevant attribute hierarchy 420 are large.

In some embodiments, one or more anomaly detection models can be used. An anomaly detection model can be used to support a machine learning model to detect an anomaly in the operation or output of the supported machine learning model. In some cases, metadata from a machine learning model can be used by the anomaly detection model for the detection of anomalies. For instance, an anomaly detection model can detect an unusual selection of an item 320 from the universal item database 310 for a graded substitutable group category 140. When an anomaly is detected, an alert can be generated by the anomaly detection model to alert a user to review the anomaly. For instance, the unusual selection of an item 320 can be presented to a physician to either confirm or deny that the selected item 320 should be included in the particular graded substitutable group category 140 for which it was selected. In some cases, users may periodically review machine learning models and anomaly detection models for accuracy.

### Systems of Products

In some embodiments, a product formulary created and utilized using the methods and systems discussed herein may also include one or more systems of items. A system of items can be a set of items 320 that are used together, such as in a single procedure. In an example, during the implantation of an ICD, a surgeon will use several other items in addition to the ICD itself, such as leads to carry electrical current from the ICD to the heart. In such an example, the ICD and the leads can be considered a system of items, in addition to any other accessories that may be used in the implantation. In another example, a hip replacement procedure can utilize a ball as well as a socket, which are separate items 320, but can be grouped together in a system due to their consistent use together in the procedure.

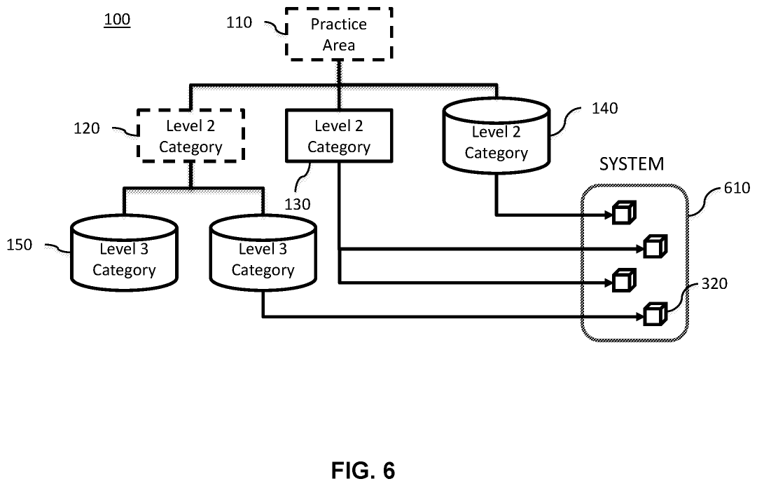

FIG. 6 illustrates an example of the clinical usage hierarchy 100 where a system of items 610 has been identified. In the example, the system of items 610 includes items 320 from three of the different categories in the clinical usage hierarchy 100. The system of items 610 includes one item 320 from the level 2 graded substitutable group category 140, two items 320 from the level 2 non-graded substitutable group category 130, and one item 320 from one of the level 3 graded substitutable group categories 150.

As another example, systems of items 610 can be created from items 320 in the clinical usage hierarchy 200 for the CRM clinical area 210 as illustrated in FIG. 2. In that example, a first system of items 610 can be created for a VVI (ventricular paced, ventricle sensed, pacemaker inhibited) ICD implantation procedure, and a second system of items 610 can be created for a BI-V (bi-ventricular) ICD implantation procedure. Both systems of items 610 can include items 320 from some of the same categories, such as from non-graded substitutable group sub-categories 260 for leads, while also included items 320 from different graded substitutable group sub-categories 250 for ICDs that are specific to the associated procedures.

In some cases, if a system of items 610 includes only a single item 320 from a graded substitutable group category 140 or 150, the system of items 610 can use a grading that is the same as the grading in the graded substitutable group category 140 or 150 where the graded item originated (e.g., having the same tiers 510). In cases where a system of items 610 includes two or more graded items, the system of items 610 can itself be graded. In such an example, the system of items 610 can be separated into groups of items inside of the system of items 610. In some cases, the groups can include only the graded items, as the non-graded items can be interchangeable by their virtue of being non-graded. A training set for a machine learning model can be created using a portion of the groups of items, such as by manual grading of the groups by a user (e.g., physician or other subject matter expert in the practice area 110) or via an automated process, such as discussed above, where the portion of the groups are graded into tiers 510. The machine learning model can be trained on the training set and then deployed on the remainder of the groups of items in order to sort the groups of items into the tiers 510.

### Data Importation in a Digital Marketplace

A product formulary can be implemented into a digital marketplace in order to provide greater benefits to physicians or healthcare providers that are selecting preferred items, such as through reduced costs and expenses. A product formulary can drive lower prices by the grouping and grading processes discussed above, which can foster competition among manufacturers and provide physicians or healthcare providers with alternative, substitutable items they may have been previously unaware of.

A digital marketplace for the selection of medical product items from a product formulary can be implemented using any suitable method, such as a cloud-based platform that is accessible via a website, an application program, using application programing interfaces, etc. As part of the onboarding process for healthcare providers to participate in the digital marketplace, the historical use of medical product items and other products in the product formularies can be entered into the appropriate product formularies. Historical usage data for items used by the healthcare provider can be mapped to categorization information, such as the same categorization information used in the universal item database 310, such as the FDA product code and GMDN. Mapping of the historical usage data can utilize any available data, such as manufacturer names, model numbers, serial numbers, and any other information located in the historical usage data, which can be used to identify the corresponding items 320 in the universal item database 310. The identified items 320 can then be entered into the appropriate categories in the clinical usage hierarchies 100 for the practice areas 110 for which the healthcare provider used the items 320.

In some cases, a direct mapping between the historical usage data and the universal item database 310 can be difficult due to incorrect or incomplete information in the historical usage data, human error, the use of alternative categorization systems, etc. For instance, the GUDID includes both model numbers and catalog numbers for items 320, while healthcare providers will often use either value interchangeably in a single model number field. When only one field is used in the historical usage data to store data that is separated in the universal item database 310, this can result in an item in the historical usage data being incorrectly mapped to a different item 320 in the universal item database 310. For example, the historical usage data can include a “model number” of 4567 for an ICD, which may be the catalog number for the ICD rather than the model number, entered into the model number field due to the conflation by the healthcare provider. In such an example, the historical usage data of the item may be attributed to an item 320 with a model number of 4567 that can be drastically different from the ICD that was used, while the ICD that was used is in the universal item database 310 with the catalog number of 4567 and a different model number.

In order to remediate errors in these types of situations, a machine learning model can be trained and used. A training set can be created using items from the historical usage data that have been successfully matched with items 320 in the universal item database 310, and, in some cases, verified as being accurate by a user (e.g., a physician or subject matter expert in the practice area 110). The training set can also include procedure-level context for items as well, which can include items in the historical usage data that are associated together, such as used in a single procedure together. The machine learning model can use the training set and be deployed on the entire set of historical usage data to match the historical usage data to the items 320 in the universal item database 310.

The use of a machine learning model can remediate the types of errors discussed above, such as by first detecting that the initial match is incorrect using anomaly detection. An incorrect match can be detected if an item included in a group of items for a procedure is anomalous (e.g., two pacemakers would not be used in a single CRM procedure), if the price for an item in the historical usage data is an outlier when compared to other prices for the same or similar items in the historical usage data and/or when compared to known prices for the item in the universal item database 310 or the product formularies, if an item is unusual in a group of items for a procedure, a combination of such processes, etc. Once the model has detected an error in the matching of an item in the historical usage data, the model can determine a correction to the match based on the training and machine learning processes. For instance, in the above example of an incorrect match for an ICD where its catalog number was mistakenly entered in the historical usage data as a model number, the machine learning model can identify an incorrect match of the historical usage item to the item 320 in the universal item database 310 with the model number of 4567 when the historical usage item was used in a procedure for an ICD implantation and the item 320 is an item not typically used in ICD implantations and the list of items for the procedure in the historical usage data, as matched, does not include an ICD. Once the model determines the match is incorrect, the model can attempt to match the “model number” in the historical usage data with catalog numbers in the universal item database 310, identify that an ICD that is compatible with the other products used in the procedure has a catalog number of 4567, and determine that the identified ICD matches other aspects of the historical usage data (e.g., via price comparisons, other uses of the ICD in other procedures, usage of other items from the same manufacturer by the same physician, etc.).

In some embodiments, the machine learning model can automatically correct the match in the mapping of the historical usage data. In other embodiments, a user can be informed of the incorrect match and, in some cases, presented with the suggested correction as identified by the machine learning model. The user can approve the match or provide their own match to an item 320 in the universal item database 310, which can then be implemented in the mapping, and can be used to further train the machine learning model for ongoing and future operations. Historical usage data, after being entered into product formularies during onboarding of a healthcare provider, can be used to enhance the participation for the healthcare provider in the digital marketplace. Historical usage data can enhance the information in product formularies, be used to advance training of the machine learning models discussed above and can also be used to create personalized grading for graded substitutable group categories 140 that are tailored to a specific physician or healthcare provider. For instance, historical usage data can indicate certain preferences for manufacturers, item materials, item sizes, etc., which can be used to grade items into tiers 510 that are specifically tailored for the preferences. In a specific example, historical usage data can indicate that a specific physician regularly purchases products in a specific graded substitutable group category 140 that have distinct ergonomic characteristics compared to other items 320 in the same graded substitutable group category 140, such as handheld tools that are specifically designed for left-handed use. In such an example, the tiers 510 for that graded substitutable group category 140, when used by that specific physician, can be modified such that handheld tools specifically designed for left-handed use are graded higher.

### Physician Preference for Procedure Items in a Digital Marketplace

Once a physician or healthcare provider has been onboarded with the digital marketplace and historical usage data entered into the product formularies, a physician or other healthcare provider, can enter a procedure into the digital marketplace and select procedure items related to the procedure aided by the product formulary for that procedure. Procedures in the digital marketplace can be organized into four different categories: planned, reported, invoiced, and closed. A procedure is categorized as planned when it is first entered by the physician (e.g., or other user on behalf of the physician), categorized as reported when the physician has entered a procedure report, categorized as invoiced when a vendor has submitted an invoice that has been matched to the procedure, and categorized as closed once prices have been matched for the procedure items for the procedure.

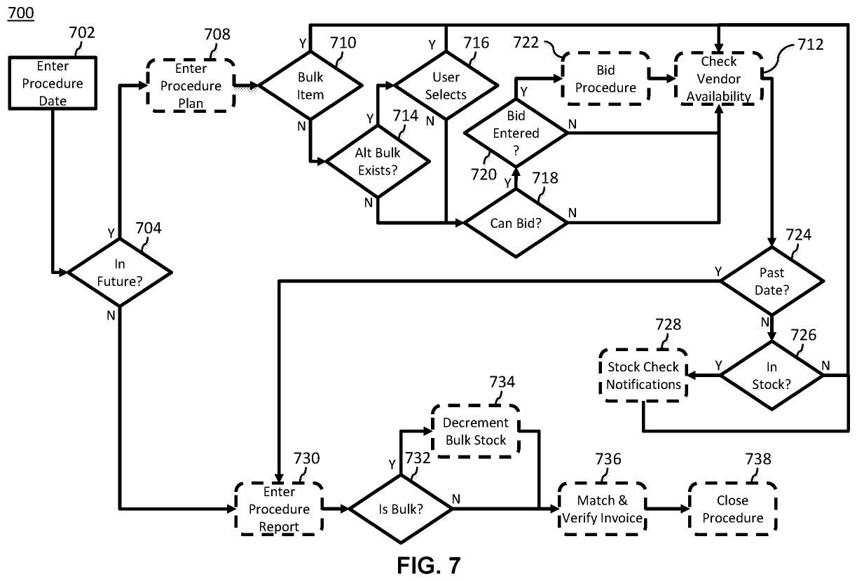

FIG. 7 illustrates a method 700 for the processing of a procedure through the digital marketplace where a product formulary is used to aid in the selection, fulfillment, and payment of medical product items for the procedure. In step 702, a procedure date is entered for the procedure by the physician along with any other required data. Minimum data requirements can vary based on the procedure, its clinical usage hierarchy 100, etc. For instance, a date, time, facility name physician name, and at least one item 320 can be required, where the level of category information for the item 320 can correspond to the level of categories in the applicable clinical usage hierarchy 100. For example, if the procedure is a CRM procedure where the clinical usage hierarchy 200 of FIG. 2 applies, each selected item 320 for the procedure should denote the category to a third level due to there being three levels in the clinical usage hierarchy 200 (e.g., if the item is an ICD, the item as entered in the procedure can include a first category of CRM practice area 210, a second category of the ICD organizational pass-through category 220, and a third category of the ICD graded substitutable group category 250 to which the selected ICD belongs). When the procedure is first entered, detailed information for the item may be optional (e.g., not required) as the actual item being used for the procedure may yet to be selected by the physician, such as if the physician is utilizing the product formulary to select a suitable item 320 from the graded substitutable group category 250 to which the item 320 belongs. In such an instance, the detailed information from the item can be entered after selection of the item, which can be done automatically by a processor of the digital marketplace, as discussed below, such as during the entering of a procedure report for the procedure.

In order to facilitate the entry of a procedure into the system in step 702, a user interface can be provided whereby the physician can enter the procedure and item data. In some cases, the user interface can provide guidance to the physician in the selection and entry of items and other details. For example, the physician can select the procedure from a list of procedures (e.g., associated with practice areas 110 for all clinical usage hierarchies 100 for which product formularies have been created), or can start to type the name of a procedure that can be matched to a procedure associated with a clinical usage hierarchy 100 and where such match can be presented to the physician as an autofill for a procedure name text field. If a known procedure is selected, if the physician attempts to add a new item or system of items to the procedure, the physician can be presented with non-graded and graded substitutable group categories 130, 140, and 150 as well as systems of items 610 for selection. In such cases, the product formulary can be used to significantly increase the efficiency in the entry of a procedure in addition to providing the physician with alternative preference items and cost savings.

Pricing data for each item for an entered procedure can be collected, which can be provided as an optional item when a procedure is initially entered. When an item is selected, the system can automatically populate an expected or average price for the item based on the data in the relevant group category in the product formulary. In some instances, such a price can be suggested by the user interface but can provide the physician with the ability to override the suggested price and enter a specific price for the item.

Once a procedure has been entered by the physician, the system can determine, in step 704, if the entered procedure is for a procedure that has already been conducted or for a planned procedure. If the procedure has already been conducted (e.g., the procedure data entered for the procedure is in the past), then the method 700 can proceed to step 730 for the entry of a procedure report, discussed in more detail below. If the procedure has not yet been conducted, then the method 700 can proceed to step 708. In step 708, the procedure can enter a planning stage where the procedure can be categorized as planned and the physician (e.g., or other user) can proceed to select specific items 320 for use in the procedure. In some cases, the physician can be provided with a list of items 320 for selection for the procedure, such as being provided with lists of items in the non-graded and graded substitutable groups categories 130 and 140 for the clinical usage hierarchy 100 for the specific procedure. In other cases, the physician can manually enter the specific items they are interested in using for the procedure. For items directly selected as part of the procedure plan, the method 700 can proceed to step 710. In some cases, the physician can be provided an opportunity to check for the possibility of selecting an item 320 via a bidding process and proceed directly to step 718 for an item 320, as discussed below.

In step 710, the system can check existing stocks of items for each item 320 in the procedure plan to determine if there are any available items in stock for the procedure. In some cases, the specific stock for the item 320 can be unique to the healthcare provider. If there is available stock for the selected item 320, then the method 700 proceeds to step 712 where the system checks with the vendor associated with the selected item 320 to determine if the item is available. The system can electronically transmit a request message to the vendor's system to determine if the item 320 is available, where such data may not be directly available to the digital marketplace system, such as if the digital marketplace system cannot interface with an inventory management system of the vendor or if the existing stock of the item 320 with the vendor are already reserved for other procedures. If the system determines, in step 710, that there is no available stock for the selected item 320, then, in step 714, the system can determine if a substitutable alternative for the item 320 exists. The system can identify substitutable alternatives using the group categories in the clinical usage hierarchy 100 for the practice area 110. In cases where the item 320 belongs to a non-graded substitutable group category 130, any other item 320 in the category 130 can be identified as a substitutable alternative. In cases where the item 320 belongs to a graded substitutable group category 140 or 150, only other items 320 in the same grade (e.g., same tier 510) as the selected item 320 can be identified as a substitutable alternative. In some instances, the system can determine that another item 320 is only a substitutable alternative if the item 320 is currently in stock. In some such instances, a check with vendor availability, such as performed in step 712, can be performed as part of the determination in step 710.

If one or more substitutable alternatives that are in stock are identified, in step 714, then, in step 716, the alternative items 320 can be presented to the physician via a suitable user interface. The physician can view all relevant information for each of the substitutable alternatives (e.g., the clinically relevant attributes for the clinically relevant attribute hierarchy 410 for the items 320 in the substitutable group category) and select the substitutable alternative item 320 for use in the procedure. If the physician selects one of the substitutable alternative items 320, the system can modify the entered procedure plan to use the substitutable alternative item 320 and proceed to step 712 to check vendor availability for the selective substitutable alternative item 320. If the physician declines all of the substitutable alternative items 320, or, in step 714, the system determines that there are no substitutable alternative items 320 in stock or available for the procedure, then the method 700 can proceed to step 718.

In step 718, the system can determine if the selected item 320 and/or a substitutable alternative item 320 can be obtained via a bidding process. In an exemplary method, the bidding process can comprise a reverse auction to be conducted for an item in the substitutable group category that includes the selected item 320 and/or substitutable alternative item 320. The availability of such a process can depend on, for instance, if there is sufficient time before the procedure date to conduct the process, there are items 320 in the substitutable group category from more than one vendor, etc. If such a process is unavailable for the selected item 320 and/or a substitutable alternative item 320, then the system can continue to check availability of the originally selected item 320 with the vendor, in step 712, or repeat the method 700 from step 710 at periodic intervals. If a bidding process is available, then, in step 720, the system can prompt the physician to enter the bidding process. The physician can decline to participate in a bidding process for the selected item 320 and/or a substitutable alternative item 320, which can move the method 700 to step 712 where the system can continue to check availability of the originally selected item 320 with the vendor, in step 712, or repeat the method 700 from step 710 at periodic intervals.

If the physician elects to enter into a bidding process, then, in step 722, the bidding process can be conducted. In an exemplary embodiment, a reverse-auction can be conducted for the selected item 320 and/or a substitutable alternative item 320. An exemplary reverse-auction process for items 320 in the product formularies discussed herein is provided in more detail, below. Once the bidding process has completed, a new item 320 for the procedure will have been selected and entered into the procedure plan. The method 700 can then proceed to step 712 where availability of the new item 320 is checked with the vendor ahead of the procedure being performed.

As part of the method 700, the system can continue to check the availability for selected items 320 for an entered procedure plan with vendors up until the entered date for the procedure, such as via an automated process communicating with inventory systems of vendors or through manual user checks with vendor representatives, for example. In cases where vendor availability changes from prior status during one of the checks, the physician can be notified of the change in availability for the associated item 320 using the user interface. During the method 700, after a vendor availability check has been performed, in step 712, the method 700 can proceed to step 724 where the system checks the current date and time with the date and time entered for the procedure. If the procedure has not yet been conducted, then, in step 726, the system can repeat a check of the current stock of the selected item 320. If the selected item 320 is still in stock, the method 700 can return to step 712 and continue to perform vendor availability checks until the procedure has been conducted. If the selected item 320 is determined to no longer be in stock, then, in step 728, the system can provide a notification message to the physician notifying the physician that the selected item 320 is no longer in stock and instructing the physician to coordinate with the vendor. In some cases, the physician can be presented with an option to select a substitutable alternative item 320 (e.g., returning to step 714 in the method 700).

Once the procedure has been conducted, as determined in step 724 or indicated in entry of the procedure as determined in step 704, then the method 700 can proceed to step 730 where the procedure is categorized in the reported category. A procedure report is entered into the system, such as via manual entry by the physician (e.g., which can be aided by the system using the procedure plan and clinical usage hierarchy 100 for the practice area 110), or by automatic generation by the system, such as using the procedure plan and clinical usage hierarchy 100 for the practice area 110, which can be confirmed by the physician via the user interface, or parsing of other data related to the procedure, such as a charge sheet, purchase order, invoice, etc. The procedure report can include entry of all of the additional data for the procedure that was unknown or unavailable at time of entry of the procedure plan. For instance, the actual items 320 used in the procedure can be selected and associated information entered into the procedure report (e.g., model numbers, catalog numbers, and other information for each item 320 as well as the price paid for the item 320). In cases where a bidding process was conducted for an item 320, in step 722, then information from the bidding process (e.g., winning vendor, fulfilled item 320, winning bid price, etc.) can be used to auto-populate the procedure report for the associated item 320.

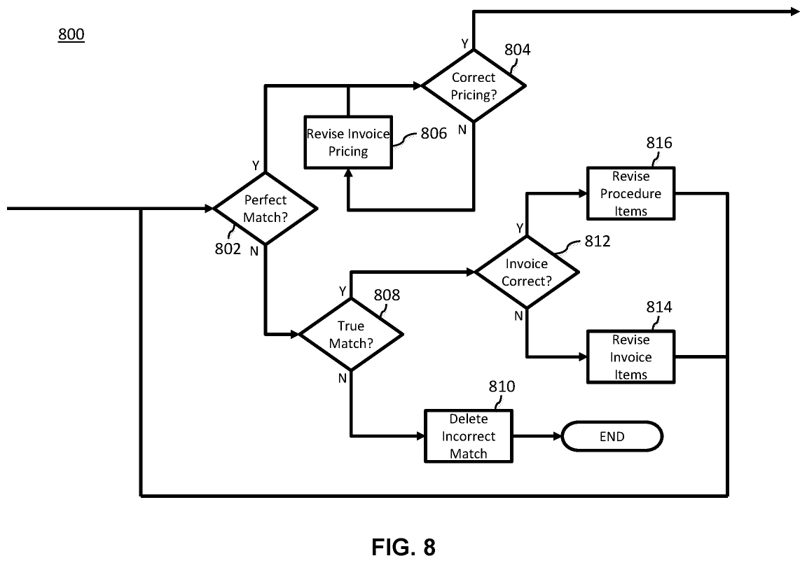

After the procedure report has been entered for the procedure, then, in step 732, the system can go through each item 320 in the procedure report to determine if the item 320 that was used is an item for which there is current stock indicated. If there is stock information for the item 320, and the stock information indicates that the item 320 is currently in stock, then, in step 734, the system can decrement the stock for the item 320. Following updating of the stock, or if no stock information for the item 320 was identified, in step 732, then the method 700 can proceed to step 736 where the system can match the conducted procedure to an invoice. A more detailed process for invoice matching conducted as step 736 is discussed in FIG. 8, below. After an invoice has been matched to the conducted procedure, the method 700 can proceed to step 738 where the procedure can be officially closed in the system. In some cases, the procedure can be closed automatically by the system after the invoice has been matched, resulting in no further activity absent specific user instructions. In other cases, the physician can be prompted to confirm closure of the procedure. In some instances, the system can require satisfaction of the invoice (e.g., confirmed payment by the healthcare provider) prior to closure of the procedure.

FIG. 8 illustrates a process 800 for the matching of an invoice to an entered procedure report as part of the digital marketplace, such as can be conducted in step 736 of the method 700 illustrated in FIG. 7. The process 800 can start at step 802, following steps 732 or 734 in FIG. 7, where the system can attempt to match a received invoice to a completed procedure. The system can determine if a perfect match can be made between an invoice and a procedure report by comparing each item and serial number on the invoice with items 320 and their corresponding serial numbers in a procedure report. In some cases, an invoice may include entries for every item 320 in a procedure report. In other cases, an invoice may only include entries for a portion of items 320 in a procedure report, such as in cases where a vendor only provides a portion of the items 320 for a specific procedure. In such cases, the process 800 can be reported for multiple invoices for a single completed procedure. If there is an exact match with the items and serials numbers of a received invoice with items 320 and serial numbers in a procedure report, then the process 800 can proceed to step 804.

In step 804, the system can compare the pricing on the invoice with pricing for the items 320 according to the product formulary for the procedure. In cases where an item 320 was selected as part of a bidding process (e.g., in step 722 of the method 700 in FIG. 7), the price of that item 320 on the invoice can be compared to the price of the winning bid in the bidding process. If any of the prices are incorrect according to the product formulary, then, in step 806, the system can revise (e.g., automatically or with confirmation from the submitting vendor) the invoice to correct the pricing. In some cases, invoice pricing will only be revised if the procedure report utilizes suggested pricing, which can enable a healthcare provider to use override pricing, as applicable. Following correction of the prices, the process 800 can return to step 804 where the correct pricing can be confirmed. Once the pricing information for the invoice is correct, the match of the invoice and the procedure can be saved in the procedure report and the process 800 completed, which can return to step 738 in the method 700 of FIG. 7.

If there is no perfect match for a received invoice to a completed procedure, as determined in step 802, then the process 800 can proceed to step 808 where the system can determine if a true match can be made between the received invoice and a completed procedure. The true match can be determined by the system using an automated process, such as by using additional invoice data or matching a significant number (e.g., above a predetermined threshold) of data points in the invoice data with the procedure report, or via a manual process. For instance, in a manual process, the physician that entered the procedure report can review the received invoice and confirm or deny if there is a true match with the received invoice and the completed procedure. In some cases, a manual review can be prompted following an automated review if the automated review indicates a true match has been found. If no true match is found between the received invoice and the completed procedure, such as due to a lack of data or a denial by the physician, then, in step 810, the system can delete the potential match, end the process 800, and start a new process 800 at step 802 for a different received invoice.

If step 808 determines there is a true match between the received invoice and the completed procedure, then, in step 812, the system can check the invoice for errors to determine if the invoice is correct or needs revisions. If the invoice is incorrect, then, in step 814, items on the invoice can be revised in order to match with the completed procedure, such as by correcting serial numbers, including omitted items, adding data to incomplete data fields, etc. If the invoice is correct (e.g., no errors in the invoice data can be identified), then, in step 816, the procedure report for the procedure can be revised, such as by correcting serial numbers in the procedure report, adding omitted items, adding data to incomplete data fields, etc. In some cases, the actions performed in step 814 and/or 816 can be performed automatically by the system, which can be entered without user confirmation or can be confirmed by the physician. In other cases, the actions performed in steps 814 and/or 816 can be performed by a physician, which can be aided by the system (e.g., revisions suggested based on the available data). Once revisions are made, the process 800 can return to step 802 where a perfect match can then be made between the invoice and procedure report and then proceed to step 804 for evaluation of the pricing.

### Reverse Auctions for Product Formularies in a Digital Marketplace

As discussed above, the digital marketplace can be configured to facilitate reverse auctions as part of a bidding process on medical product items for a procedure, such as can be conducted as part of the bidding procedure in step 722 of the method 700 of FIG. 7. While the methods and systems discussed herein describe a reverse auction, other suitable types of auctions and other bidding processes can be utilized for the selection of items 320 for a procedure and fulfillment from vendors.

In traditional marketplaces, each vendor can offer items 320 produced by that vendor at set pricing, where a physician can select items 320 at the pricing set by the vendor. Such selections can be made in the digital marketplace discussed herein, where all such items 320 and pricing information can be presented when items 320 are selected in a procedure plan. The process can be more efficient than in traditional marketplaces as a particular item 320 can be presented along with all other substitutable alternatives as graded or otherwise included in the same substitutable group category in the product formulary for the procedure being planned. This can also encourage vendors to be more competitive in pricing due to the transparency of pricing information for all alternative products.

The digital marketplace can further improve the selection process for items 320 for a procedure by the use of a reverse auction facilitated using the product formulary. When a physician is interested in conducting a bidding process for an item 320, the physician can utilize the user interface of the digital marketplace to enter information that can be used to create the reverse auction. The information can include a start date, duration, reserve price (e.g., if desired), buyout price (e.g., if desired), quantity, etc. In cases where a bulk purchase is being made (e.g., for multiple procedures for future use), the system can identify a suggested amount for the quantity based on the historical usage data for the healthcare provider. The physician can also select the specific category and, if applicable, grade in the specific category, for which an item is being requested. For instance, if the procedure being planned is a CRM procedure that is in the CRM practice area 210 of the clinical usage hierarchy 200 illustrated in FIG. 2, the physician can request an item 320 from the first pacemaker graded substitutable group sub-category 250 under the pacemaker organizational pass-through category 220. By selecting a category, only items 320 are substitutable for the specific needs of that procedure, as set forth in the product formulary, can be used in a bid for the auction, to ensure that the physician can only receive items that are suitable for use in the procedure.

In some cases, a reverse auction or other bidding process can be initiated for a specific substitutable group category without being tied to a specific procedure. For instance, a healthcare provider can utilize a reverse auction to make a bulk purchase of procedure items for use in future procedures. In such instances, the system can keep track of the stock of the pre-purchased procedure items for selection by physicians (e.g., or other users on behalf of physicians) in future procedures. For example, the inventory checks performed by the system in steps 710 and 714 can check pre-purchased stock of items 320 made through bidding processes discussed herein. Use of reverse auctions and other bidding processes, combined with the method 700 and use of a product formulary, can provide healthcare providers with significant cost savings for procedure items while enabling physicians access to suitable procedure items for any procedure.

In some instances, the physician can be presented with additional options upon the selection of a category. For instance, if the category is graded, the physician can further select desired and/or excluded tiers 510. In another example, the physician can be provided with a list of all items 320 in the category to request bids for specific items 320 or exclude specific items 320 for bids. In some cases, the system can utilize data from prior auctions (e.g., conducted for all healthcare providers, similar healthcare providers, the same physician, a combination thereof, etc.) to suggest selections by the physician, such as a recommended buyout price, recommended reserve amount, etc. In some instances, the auction process can enable the physician to select a system of items 610 to be bid on by vendors using the same processes as for individual items 320 as discussed herein.

Once all of the selections for the auction have been made, the physician can submit the information in the user interface and the system can create the auction. The system can identify all vendors registered with the system that sell at least one item 320 that is included in the selected category and not excluded by the physician and notify each of the vendors of the auction. Each vendor can then access a user interface of the digital marketplace to make a bid for the auction. The vendor can review details of the auction (e.g., duration, product category, list of eligible items 320, buyout price, etc.) and can decide whether to place a bid. If the vendor is interested in placing a bid, the vendor can select the item 320 that they are interested in selling from a list of eligible items 320 for the selected category, input any additional detail required for the selected item 320 (e.g., a serial number), provide a price, and submit the bid. If a buyout price is available, the vendor can select to pay the buyout price and immediately win the auction, which can thereby be processed, as discussed below. In some cases, the system can provide the vendor with assistance with their bid, such as recommending pricing based on historical usage data for the healthcare provider, other auctions for items 320 in the category, past bid selections by the physician, etc.

Once the duration of the auction has passed after its start date, the physician can utilize the user interface of the digital marketplace to review all of the bids and select a winning bid. In some cases, the system can automatically identify a winning bid based on criteria selected by the physician (e.g., lowest cost, preferred item, combination of multiple criteria, etc.). Once the physician selects the winning bid, the vendor can be notified and then proceed with fulfilling the bid. The vendor can identify the item 320 that was indicated in the bid, and physically deliver the appropriate quantity of the item 320 to the healthcare provider for use in the planned procedure or in future procedures, such as in the case of a bulk pre-purchase of items. The vendor can input the specific serial numbers for the delivered item(s) into the system via the user interface, where the system can store the serial numbers in its stock data for use in assisting with the entering of procedure reports, selection of items in new procedures, and determining costs in procedure reports if an item 320 purchased in a bidding process was used in an entered procedure. The system can store the information for the auction, which can be tied to the specific procedure plan, such that information for the procedure report once the procedure has been conducted can be automatically populated using the auction information (e.g., vendor name, item price, item serial number, etc.).

### Additional Benefits of the Product Formulary and Digital Marketplace

The methods and systems discussed herein provide for a variety of benefits to healthcare providers, physicians, vendors, and others through the use of a product formulary and substitutable group categories as well as the ability for a digital marketplace that provides convenience for the entering and tracking of procedure as well as procurement of medical procedure items and substitutable items using the processes discussed above. There are additional benefits that can be provided using the methods and systems discussed herein, such as providing support for the recall of medical procedure items. It is well known that medical product items fail. And, when they do, the manufacturer will issue a product recall. In some cases, the product recall obligation is on the manufacturer; however, not in every case. The system, having captured patient and medical product item information with its basis in a product formulary can provide reporting on product recalls to the physician or healthcare provider. For example, recall information for a specific item 320 or for specific serial numbers of the item 320, can be provided to the system. The system can then identify each procedure where that specific item 320 (e.g., or the specific serial numbers of that item 320) was used or is planned for use, or identify each healthcare provider that purchased the specific item 320 for future use via a bidding process. The system can then notify the healthcare provider, physician, or other suitable personnel of the recall. This capability enables the physician or healthcare provider the ability to follow-up with a patient that may have a medical product item implanted or in active use. This supports the physician or healthcare providers in their due diligence in following up with patients.

Another benefit that can be provided via the methods and systems discussed herein is the ability to create and enact a digital group purchasing organization (GPO). In a preferred embodiment, the digital marketplace can not only foster and guide lower prices through one-to-one transactions (e.g., enabling bulk bidding or spot purchasing on a participant-level basis), but can also provide price transparency to drive across-the-board price savings. In conjunction with objective attribute data a digital GPO can be enacted that provides for a variety of benefits for healthcare providers and other participants. For example, a digital GPO supported using the product formulary can facilitate collaborative procurement initiatives within the platform, allowing members to join forces for bulk purchases beyond traditional volume discounts, targeting specific needs or innovations, the system can develop algorithms to offer personalized discount programs for members, factoring in historical purchase volumes, real-time analytics can be implemented by the for spend management, enabling predictive forecasting to optimize purchasing decisions based on market trends, etc. In some instances, the use of an artificial intelligence engine for the creation of the product formulary can also be leveraged for benefits for a digital GPO, such as to automatically identify optimal pricing and negotiate contracts with vendors based on the aggregated purchasing power of the GPO, securing the best possible terms and prices for all members.

Another benefit of the methods and systems discussed herein is the ability to increase the efficiency of healthcare providers and physicians. When a procedure plan is entered, one of the required data elements is the physician who performs the procedure. This allows the digital marketplace to analyze relative doctor expenditure, which can be used to make recommendations to help lower costs. For example, a physician with very high utilization of high-grade items within a given category in a clinical usage hierarchy 110 can be recommended other items 320 in the same graded substitutability group 140 that are in the same tier 510 and cost less, other items 320 in a higher tier 510 that cost less, etc., thus reducing cost independent of lowering pricing. In addition, a healthcare provider can be notified of the numbers of items 320 used by physicians for that healthcare provider across each substitutable group category to enable the healthcare provider to bulk-purchase items for that category, which can provide savings, which can be further increased through the use of the reverse auction and other bidding processes discussed herein. Analysis of doctor item utilization can be combined with analysis of patient outcomes to derive deeper insights about the relationship between item selection and outcome and guide optimal item selection. For instance, one of the required data elements for a procedure can be the facility at which a procedure will be performed. This can allow for a single participant in the digital marketplace with multiple facilities to do granular comparative analysis between those facilities. The digital marketplace can provide “report cards” for participants to learn from differences between their facilities and make recommendations on a facility-level basis for cost efficiencies. In addition, because there are many participants within the marketplace, it can be possible to do a comparison between participants

Additional benefits can be provided to healthcare providers, physicians, patients, vendors, and other participants through the use of metrics and analytics resulting from the data gathered and used to create the product formularies as well as the additional data that can be captured via procedure planning and reporting. For instance, the system can receive data regarding clinical outcomes and, when combined with procedure item selection data, can ensure device selections can be made in conjunction with proven patient outcomes. In addition, patients can provide consent for the building of a profile based on medical history and genetic information to refine device recommendations, ensuring a highly personalized and optimal match. The system can also be used to develop an interactive platform that allows physicians to simulate different scenarios based on the recommended devices, providing insights into potential outcomes before making a final decision, implement a feedback mechanism where post-implantation outcomes can contribute to the learning of the artificial intelligence (AI) engine, continuously refining future recommendations, integrate feedback and insights from a diverse range of medical specialties into the AI model, ensuring comprehensive evaluation of devices from multiple clinical perspectives, leverage machine learning to analyze compatibility between patient profiles and device materials or technologies, reducing the risk of adverse reactions, create a platform for physicians to discuss device performance and share experiences, enriching the AI's database with real-world insights, introduce filters that consider ethical implications and legal compliance of devices, ensuring that recommendations meet all regulatory standards and ethical guidelines, develop a feature that continuously scans the healthcare technology landscape, incorporating the latest innovations into technology selection recommendations, introduce a system for collecting and analyzing feedback from Healthcare Providers on technology performance, enriching the recommendation engine with practical insights, etc.

### High Level System Architecture

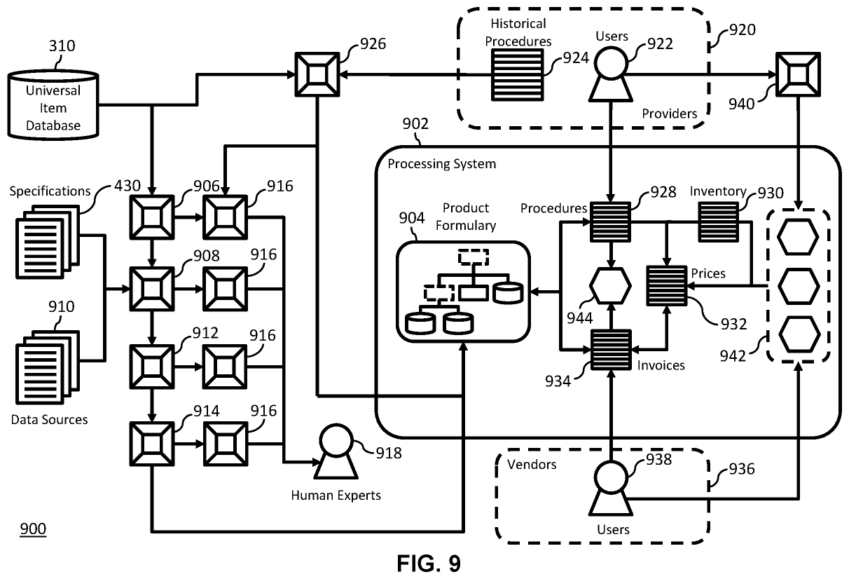

FIG. 9 illustrates a system 900 representative of a high-level system architecture for the creation and maintenance of product formularies and use thereof in the operation of a digital marketplace to perform the methods and systems as discussed herein. It will be apparent to persons having skill in the relevant art that the architecture illustrated in FIG. 9 is an example and that other suitable architectures can also be used to implement the methods and systems discussed herein.

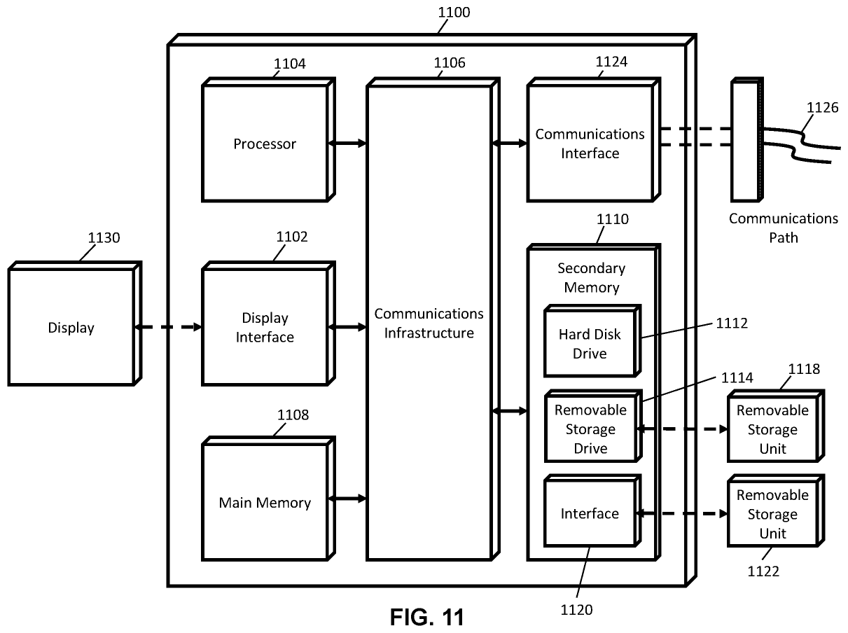

The system 900 can include a processing system 902. The processing system 902 can be a computing system, such as the computer system 1100 illustrated in FIG. 11 and discussed in more detail below, that is specially configured to perform the functions discussed herein. The processing system 902 can be configured, for example, to operate the systems perform the methods as illustrated in FIGS. 1-8, such as for the training and deployment of machine learning models, operation of artificial intelligence engines for use with machine learning models, creation of product formularies, population of non-graded and graded substitutable group categories, identification of clinically relevant attribute hierarchies, designation of clinically relevant attributes, operation of a digital marketplace, conducting of reverse auction bidding processes, matching of received invoices to procedure reports, grading of items in substitutable group categories, etc.

The processing system can include a product formulary 904, generated using the processes discussed herein. Creation of the product formulary 904 is also illustrated at a high level in the system 900. Process 906 represents the creation of the clinical usage hierarchy 100 using the universal item database 310 and a trained machine learning model, such as described above with respect to FIGS. 1-3. After the clinical usage hierarchy 100 is created, process 908 utilizes another trained machine learning model to populate clinically relevant attribute hierarchies 410 for items 320 in the various substitutable group categories for each practice area 110 using data obtained from specifications 430 and other available data sources 910, such as described above with respect to FIG. 4. Other available data sources can include any additional databases and/or computing systems that have data that can be collected by, requested by, or otherwise provided to the processing system 902 as part of the methods and systems discussed herein. Data sources 910 can include item manufacturers, product specification databases, data tracking agencies, healthcare providers, medical research agencies, regulatory bodies, governmental agencies, etc.

Process 912 illustrated in the system 900 can include the grading of items 320 in the graded substitutable group categories 140 and 150 of the product formularies 904, such as described above with respect to FIG. 5. In process 914, systems of items 610 can be created for groups of associated items 320 in the clinical areas 110, such as described above with respect to FIG. 6. The system 900 can also include anomaly detection processes 916, which can be used to detect anomalies in the processes 906, 908, 912, and 914, such as in the operation of trained machine learning models and anomalous outputs. In some embodiments, results of the anomaly detection processes 916 can be reviewed by one or more human experts 918, who can review detected anomalies, adjust category selections, clinically relevant attributes, item grading, etc., and modify machine learning models to correct anomalies.

The system 900 can also include one or more healthcare providers 920. Healthcare providers 920 can be computing systems or other devices associated with entities that participate in the system 900 to provide data for the building of product formularies and usage of product formularies. Healthcare providers 920 can be onboarded by the processing system 902 for participation in a digital marketplace to make use of the product formularies 904 using the methods discussed herein. Healthcare providers 920 can include a plurality of users 922, such as physicians or healthcare providers. As part of an onboarding process 926, historical usage data 924 for the healthcare provider 920 can be submitted into the processing system and included in relevant product formularies 904, such as using the process discussed above. The users 922 of the healthcare provider 920 can then utilize the processing system 902 to enter procedure data for medical procedures, select items 320 for procedures, elicit bidding from item suppliers, etc., as discussed herein.

The processing system 902 can include various data types, which can be stored in any suitable data formatting methods and schema and can be any suitable type of memory, such as read-only memory, random access memory, etc. In some embodiments, the data storage can be comprised of or can otherwise include a relational database that utilizes structured query language for the storage, identification, modifying, updating, accessing, etc. of structured data sets stored therein. The processing system 902 can store procedure data 928, which can include procedure plans, procedure reports, selected items 320, information on bidding processes for selected items, etc., such as discussed above in more detail with respect to FIG. 7. The processing system 902 can also store inventory data 930, which can include stock information for items 320, which can be specific to healthcare providers 920, specific to vendors 936, and be updated as part of the processes discussed herein, such as from the selection and use of items 320 in procedure plans and procedure reports.

The processing system 902 can also store price data 932. The price data 932 can be included in product formularies 904 as pricing information and histories for items 320 that are available for selection by users 922 for newly entered medical procedures. The processing system 902 can also store invoice data 934 for invoices entered by vendors 936. As discussed above, invoice data 934 can be submitted and can be matched by the processing system 902 to existing procedure reports for use in updating inventory data 930, procedure data 928, and price data 932, such as using the matching process illustrated in FIG. 8 and discussed above. The system 900 can include one or more vendors 936 that supply the invoice data 934 via one or more users 938 thereof that can utilize suitable computing systems and devices in order to interface with the processing system 902 using suitable communication networks and methods. The processing system 902 is also configured to operate bidding processes 942. In a process 940, a user 922 of a healthcare provider 920 can initiate a bidding process 942, such as a reverse auction as discussed in more detail above, to enable vendors 936 to enter bids to supply one or more items 320 for a planned procedure. Users 938 of the vendors 936 can enter bids, which can be accepted or denied by the users 922 of the healthcare providers 920 and provide healthcare providers 920 with a cost effective and efficient process for obtaining items 920 for medical procedures and identify substitutable items 320 for additional options that can provide greater efficiency and reduce costs due to diversity of choices and fostered competition among vendors 936.

### Exemplary Method for Product Formulary Creation

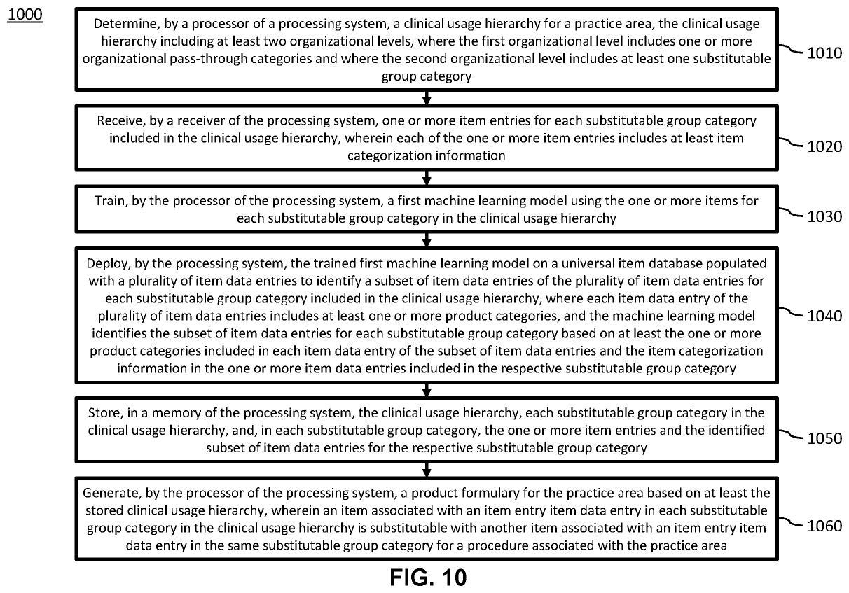

FIG. 10 illustrates a method 1000 for the creation of a product formulary for medical product items (e.g., physician preference items) utilizing machine learning and substitutable group categories for a clinical usage hierarchy.

In step 1010, a clinical usage hierarchy (e.g., clinical usage hierarchy 100) can be determined by a processor of a processing system (e.g., processing system 910) for a practice area, the clinical usage hierarchy including at least two organizational levels, where the first organizational level includes one or more organizational pass-through categories (e.g., organizational pass-through categories 120) and where the second organizational level includes at least one substitutable group category (e.g., substitutable group categories 130, 140, 150, etc.). In step 1020, one or more item entries can be received by a receiver of the processing system for each substitutable group category included in the clinical usage hierarchy, wherein each of the one or more item entries includes at least item categorization information.

In step 1030, a first machine learning model can be trained by the processor of the processing system using the one or more items for each substitutable group category in the clinical usage hierarchy. In step 1040, the trained first machine learning model can be deployed by the processing system on a universal item database (e.g., universal item database 310) populated with a plurality of item data entries (e.g., items 320) to identify a subset of item data entries of the plurality of item data entries for each substitutable group category included in the clinical usage hierarchy, where each item data entry of the plurality of item data entries includes at least one or more product categories, and the machine learning model identifies the subset of item data entries for each substitutable group category based on at least the one or more product categories included in each item data entry of the subset of item data entries and the item categorization information in the one or more item data entries included in the respective substitutable group category.

In step 1050, the clinical usage hierarchy, each substitutable group category in the clinical usage hierarchy, and, in each substitutable group category, the one or more item entries and the identified subset of item data entries for the respective substitutable group category can be stored by the processing system in a memory of the processing system. In step 1060, the processor of the processing system can generate a product formulary for the practice area based on at least the stored clinical usage hierarchy, wherein an item associated with an item entry item data entry in each substitutable group category in the clinical usage hierarchy is substitutable with another item associated with an item entry item data entry in the same substitutable group category for a procedure associated with the practice area.

In one embodiment, the method 1000 can further include: identifying, by the processor of the processing system, a clinically relevant attribute hierarchy (e.g., clinically relevant attribute hierarchy 410) for each substitutable group category in the clinical usage hierarchy, the clinically relevant attribute hierarchy including a hierarchy of a plurality of clinically relevant attributes for items associated with the respective substitutable group category; populating, by the processing system, the clinically relevant attribute hierarchy for two or more items (e.g., subset 410) associated with each substitutable group category in the clinical usage hierarchy with a set of clinically relevant attributes identified using one or more available data sources (e.g., specifications 430); training, by the processor of the processing system, a second machine learning model using the populated clinically relevant attribute hierarchies for the two or more items associated with each substitutable group category in the clinical usage hierarchy; deploying, by the processing system, the trained second machine learning model on the one or more item entries and subset of item data entries included in each substitutable group category in the clinical usage hierarchy to populate, for each of the one or more item entries and subset of item data entries, the clinically relevant attribute hierarchy for an associated item; and storing, in the memory of the processing system, the populated clinically relevant attribute hierarchy for the one or more item entries and subset of item data entries included in each substitutable group category in the clinical usage hierarchy, wherein the generated product formulary is further based on the populated clinically relevant attribute hierarchy for the one or more item entries and subset of item data entries included in each substitutable group category in the clinical usage hierarchy. In some embodiments, the universal item database can be a Global Unique Device Identification Database (GUDID).

In one embodiment, at least one of the substitutable group categories in the clinical usage hierarchy can be a graded substitutable group category (e.g., graded substitutable group category 140 or 150), and the one or more item entries and subset of item data entries included in the graded substitutable group category can be organized into two or more graded tiers (e.g., tiers 510). In a further embodiment, the method 1000 can further include deploying, by the processing system, a third machine learning model on the one or more item entries and subset of item data entries included in the graded substitutable group category to organize the one or more item entries and subset of item data entries into the two or more graded tiers. In an even further embodiment, the method 1000 can also include: receiving, by the receiver of the processing system, a tier assignment to one of the two or more graded tiers for each of at least one of the item data entries (e.g., subset of graded items 520) in the subset of item data entries included in the graded substitutable group category; and training, by the processing system, the third machine learning model on the at least one of the item data entries in the subset of item data entries included in the graded substitutable group category based on the received tier assignment.

In some embodiments, the method 1000 can further include transmitting, by a transmitter of the processing system, a user interface to a user device (e.g., user device 940) in communication with the processing system, that displays a plurality of form elements for entry of procedure data for a medical procedure associated with the practice area, wherein the plurality of form elements includes at least one form element for selection of an item associated with at least one of the substitutable group categories in the clinical usage hierarchy, and the user interface pre-populates one or more data values for the at least one form element based on the generated product formulary. In one embodiment, the processing system can include an artificial intelligence engine, and the artificial intelligence engine can be configured to deploy the first trained machine learning model.

### Computer System Architecture

FIG. 11 illustrates a computer system 1100 in which embodiments of the present disclosure, or portions thereof, can be implemented as computer-readable code. For example, the processing system 910, data sources 920, healthcare provider 930, user device 940, and vendors 950 of FIG. 9 can be implemented in the computer system 1100 using hardware, non-transitory computer readable media having instructions stored thereon, or a combination thereof and can be implemented in one or more computer systems or other processing systems. Hardware can embody modules and components used to implement the systems and methods of FIGS. 1-10.

If programmable logic is used, such logic can execute on a commercially available processing platform configured by executable software code to become a specific purpose computer or a special purpose device (e.g., programmable logic array, application-specific integrated circuit, etc.). A person having ordinary skill in the art can appreciate that embodiments of the disclosed subject matter can be practiced with various computer system configurations, including multi-core multiprocessor systems, minicomputers, mainframe computers, computers linked or clustered with distributed functions, as well as pervasive or miniature computers that can be embedded into virtually any device. For instance, at least one processor device and a memory can be used to implement the above-described embodiments.

A processor unit or device as discussed herein can be a single processor, a plurality of processors, or combinations thereof. Processor devices can have one or more processor “cores.” The terms “computer program medium,” “non-transitory computer readable medium,” and “computer usable medium” as discussed herein are used to generally refer to tangible media such as a removable storage unit 1118, a removable storage unit 1122, and a hard disk installed in hard disk drive 1112.

Various embodiments of the present disclosure are described in terms of this example computer system 1100. After reading this description, it will become apparent to a person skilled in the relevant art how to implement the present disclosure using other computer systems and/or computer architectures. Although operations can be described as a sequential process, some of the operations can in fact be performed in parallel, concurrently, and/or in a distributed environment, and with program code stored locally or remotely for access by single or multi-processor machines. In addition, in some embodiments the order of operations can be rearranged without departing from the spirit of the disclosed subject matter.

Processor device 1104 can be a special purpose or a general-purpose processor device specifically configured to perform the functions discussed herein. The processor device 1104 can be connected to a communications infrastructure 1106, such as a bus, message queue, network, multi-core message-passing scheme, etc. The network can be any network suitable for performing the functions as disclosed herein and can include a local area network (LAN), a wide area network (WAN), a wireless network (e.g., WiFi), a mobile communication network, a satellite network, the Internet, fiber optic, coaxial cable, infrared, radio frequency (RF), or any combination thereof. Other suitable network types and configurations will be apparent to persons having skill in the relevant art. The computer system 1100 can also include a main memory 1108 (e.g., random access memory, read-only memory, etc.), and can also include a secondary memory 1110. The secondary memory 1110 can include the hard disk drive 1112 and a removable storage drive 1114, such as a floppy disk drive, a magnetic tape drive, an optical disk drive, a flash memory, etc.

The removable storage drive 1114 can read from and/or write to the removable storage unit 1118 in a well-known manner. The removable storage unit 1118 can include a removable storage media that can be read by and written to by the removable storage drive 1114. For example, if the removable storage drive 1114 is a floppy disk drive or universal serial bus port, the removable storage unit 1118 can be a floppy disk or portable flash drive, respectively. In one embodiment, the removable storage unit 1118 can be non-transitory computer readable recording media.

In some embodiments, the secondary memory 1110 can include alternative means for allowing computer programs or other instructions to be loaded into the computer system 1100, for example, the removable storage unit 1122 and an interface 1120. Examples of such means can include a program cartridge and cartridge interface (e.g., as found in video game systems), a removable memory chip (e.g., EEPROM, PROM, etc.) and associated socket, and other removable storage units 1122 and interfaces 1120 as will be apparent to persons having skill in the relevant art.

Data stored in the computer system 1100 (e.g., in the main memory 1108 and/or the secondary memory 1110) can be stored on any type of suitable computer readable media, such as optical storage (e.g., a compact disc, digital versatile disc, Blu-ray disc, etc.) or magnetic tape storage (e.g., a hard disk drive). The data can be configured in any type of suitable database configuration, such as a relational database, a structured query language (SQL) database, a distributed database, an object database, etc. Suitable configurations and storage types will be apparent to persons having skill in the relevant art.

The computer system 1100 can also include a communications interface 1124. The communications interface 1124 can be configured to allow software and data to be transferred between the computer system 1100 and external devices. Exemplary communications interfaces 1124 can include a modem, a network interface (e.g., an Ethernet card), a communications port, a PCMCIA slot and card, etc. Software and data transferred via the communications interface 1124 can be in the form of signals, which can be electronic, electromagnetic, optical, or other signals as will be apparent to persons having skill in the relevant art. The signals can travel via a communications path 1126, which can be configured to carry the signals and can be implemented using wire, cable, fiber optics, a phone line, a cellular phone link, a radio frequency link, etc.

The computer system 1100 can further include a display interface 1102. The display interface 1102 can be configured to allow data to be transferred between the computer system 1100 and external display 1130. Exemplary display interfaces 1102 can include high-definition multimedia interface (HDMI), digital visual interface (DVI), video graphics array (VGA), etc. The display 1130 can be any suitable type of display for displaying data transmitted via the display interface 1102 of the computer system 1100, including a cathode ray tube (CRT) display, liquid crystal display (LCD), light-emitting diode (LED) display, capacitive touch display, thin-film transistor (TFT) display, etc.

Computer program medium and computer usable medium can refer to memories, such as the main memory 1108 and secondary memory 1110, which can be memory semiconductors (e.g., DRAMs, etc.). These computer program products can be means for providing software to the computer system 1100. Computer programs (e.g., computer control logic) can be stored in the main memory 1108 and/or the secondary memory 1110. Computer programs can also be received via the communications interface 1124. Such computer programs, when executed, can enable computer system 1100 to implement the present methods as discussed herein. In particular, the computer programs, when executed, can enable processor device 1104 to implement the systems and methods illustrated by FIGS. 1-10, as discussed herein. Accordingly, such computer programs can represent controllers of the computer system 1100. Where the present disclosure is implemented using software, the software can be stored in a computer program product and loaded into the computer system 1100 using the removable storage drive 1114, interface 1120, and hard disk drive 1112, or communications interface 1124.

The processor device 1104 can comprise one or more modules or engines configured to perform the functions of the computer system 1100. Each of the modules or engines can be implemented using hardware and, in some instances, can also utilize software, such as corresponding to program code and/or programs stored in the main memory 1108 or secondary memory 1110. In such instances, program code can be compiled by the processor device 1104 (e.g., by a compiling module or engine) prior to execution by the hardware of the computer system 1100. For example, the program code can be source code written in a programming language that is translated into a lower-level language, such as assembly language or machine code, for execution by the processor device 1104 and/or any additional hardware components of the computer system 1100. The process of compiling can include the use of lexical analysis, preprocessing, parsing, semantic analysis, syntax-directed translation, code generation, code optimization, and any other techniques that can be suitable for translation of program code into a lower-level language suitable for controlling the computer system 1100 to perform the functions disclosed herein. It will be apparent to persons having skill in the relevant art that such processes result in the computer system 1100 being a specially configured computer system 1100 uniquely programmed to perform the functions discussed above.

Techniques consistent with the present disclosure provide, among other features, systems and methods for creation of a product formulary for medical product items). While various exemplary embodiments of the disclosed system and method have been described above it should be understood that they have been presented for purposes of example only, not limitations. It is not exhaustive and does not limit the disclosure to the precise form disclosed. Modifications and variations are possible in light of the above teachings or can be acquired from practicing of the disclosure, without departing from the breadth or scope.

What is claimed is:

1. A method for creation of a product formulary for medical product items, comprising:
determining, by a processor of a processing system, a clinical usage hierarchy for a practice area, the clinical usage hierarchy including at least two organizational levels, where the first organizational level includes one or more organizational pass-through categories and where the second organizational level includes at least one substitutable group category;
receiving, by a receiver of the processing system, one or more item entries for each substitutable group category included in the clinical usage hierarchy, wherein each of the one or more item entries includes at least item categorization information;
training, by the processor of the processing system, a first machine learning model using the one or more items for each substitutable group category in the clinical usage hierarchy;
deploying, by the processing system, the trained first machine learning model on a universal item database populated with a plurality of item data entries to identify a subset of item data entries of the plurality of item data entries for each substitutable group category included in the clinical usage hierarchy, where each item data entry of the plurality of item data entries includes at least one or more product categories, and the machine learning model identifies the subset of item data entries for each substitutable group category based on at least the one or more product categories included in each item data entry of the subset of item data entries and the item categorization information in the one or more item data entries included in the respective substitutable group category;
storing, in a memory of the processing system, the clinical usage hierarchy, each substitutable group category in the clinical usage hierarchy, and, in each substitutable group category, the one or more item entries and the identified subset of item data entries for the respective substitutable group category; and
generating, by the processor of the processing system, a product formulary for the practice area based on at least the stored clinical usage hierarchy, wherein an item associated with an item entry item data entry in each substitutable group category in the clinical usage hierarchy is substitutable with another item associated with an item entry item data entry in the same substitutable group category for a procedure associated with the practice area.

2. The method of claim 1, further comprising:
identifying, by the processor of the processing system, a clinically relevant attribute hierarchy for each substitutable group category in the clinical usage hierarchy, the clinically relevant attribute hierarchy including a hierarchy of a plurality of clinically relevant attributes for items associated with the respective substitutable group category;
populating, by the processing system, the clinically relevant attribute hierarchy for two or more items associated with each substitutable group category in the clinical usage hierarchy with a set of clinically relevant attributes identified using one or more available data sources;
training, by the processor of the processing system, a second machine learning model using the populated clinically relevant attribute hierarchies for the two or more items associated with each substitutable group category in the clinical usage hierarchy;
deploying, by the processing system, the trained second machine learning model on the one or more item entries and subset of item data entries included in each substitutable group category in the clinical usage hierarchy to populate, for each of the one or more item entries and subset of item data entries, the clinically relevant attribute hierarchy for an associated item; and
storing, in the memory of the processing system, the populated clinically relevant attribute hierarchy for the one or more item entries and subset of item data entries included in each substitutable group category in the clinical usage hierarchy,
wherein the generated product formulary is further based on the populated clinically relevant attribute hierarchy for the one or more item entries and subset of item data entries included in each substitutable group category in the clinical usage hierarchy.

3. The method of claim 1, wherein the universal item database is a Global Unique Device Identification Database (GUDID).

4. The method of claim 1, wherein
at least one of the substitutable group categories in the clinical usage hierarchy is a graded substitutable group category, and
the one or more item entries and subset of item data entries included in the graded substitutable group category are organized into two or more graded tiers.

5. The method of claim 4, further comprising:
deploying, by the processing system, a third machine learning model on the one or more item entries and subset of item data entries included in the graded substitutable group category to organize the one or more item entries and subset of item data entries into the two or more graded tiers.

6. The method of claim 5, further comprising:
receiving, by the receiver of the processing system, a tier assignment to one of the two or more graded tiers for each of at least one of the item data entries in the subset of item data entries included in the graded substitutable group category; and
training, by the processing system, the third machine learning model on the at least one of the item data entries in the subset of item data entries included in the graded substitutable group category based on the received tier assignment.

7. The method of claim 1, further comprising:
transmitting, by a transmitter of the processing system, a user interface to a user device in communication with the processing system, that displays a plurality of form elements for entry of procedure data for a medical procedure associated with the practice area, wherein
the plurality of form elements includes at least one form element for selection of an item associated with at least one of the substitutable group categories in the clinical usage hierarchy, and
the user interface pre-populates one or more data values for the at least one form element based on the generated product formulary.

8. The method of claim 1, wherein
the processing system includes an artificial intelligence engine, and
the artificial intelligence engine is configured to deploy the first trained machine learning model.

9. A system for creation of a product formulary for medical product items, comprising:
one or more processors; and
a non-transitory computer readable medium storing instructions when executed by the one or more processors cause the one or more processors to:
determine a clinical usage hierarchy for a practice area, the clinical usage hierarchy including at least two organizational levels, where the first organizational level includes one or more organizational pass-through categories and where the second organizational level includes at least one substitutable group category;
receive one or more item entries for each substitutable group category included in the clinical usage hierarchy, wherein each of the one or more item entries includes at least item categorization information;
train a first machine learning model using the one or more items for each substitutable group category in the clinical usage hierarchy;
deploy the trained first machine learning model on a universal item database populated with a plurality of item data entries to identify a subset of item data entries of the plurality of item data entries for each substitutable group category included in the clinical usage hierarchy, where each item data entry of the plurality of item data entries includes at least one or more product categories, and the machine learning model identifies the subset of item data entries for each substitutable group category based on at least the one or more product categories included in each item data entry of the subset of item data entries and the item categorization information in the one or more item data entries included in the respective substitutable group category;
store, in a memory, the clinical usage hierarchy, each substitutable group category in the clinical usage hierarchy, and, in each substitutable group category, the one or more item entries and the identified subset of item data entries for the respective substitutable group category; and
generate a product formulary for the practice area based on at least the stored clinical usage hierarchy, wherein an item associated with an item entry item data entry in each substitutable group category in the clinical usage hierarchy is substitutable with another item associated with an item entry item data entry in the same substitutable group category for a procedure associated with the practice area.

10. The system of claim 9, wherein the instructions when executed by the one or more processors further cause the one or more processors to:
identify a clinically relevant attribute hierarchy for each substitutable group category in the clinical usage hierarchy, the clinically relevant attribute hierarchy including a hierarchy of a plurality of clinically relevant attributes for items associated with the respective substitutable group category;
populate the clinically relevant attribute hierarchy for two or more items associated with each substitutable group category in the clinical usage hierarchy with a set of clinically relevant attributes identified using one or more available data sources;
train a second machine learning model using the populated clinically relevant attribute hierarchies for the two or more items associated with each substitutable group category in the clinical usage hierarchy;
deploy the trained second machine learning model on the one or more item entries and subset of item data entries included in each substitutable group category in the clinical usage hierarchy to populate, for each of the one or more item entries and subset of item data entries, the clinically relevant attribute hierarchy for an associated item; and
store, in the memory, the populated clinically relevant attribute hierarchy for the one or more item entries and subset of item data entries included in each substitutable group category in the clinical usage hierarchy,
wherein the generated product formulary is further based on the populated clinically relevant attribute hierarchy for the one or more item entries and subset of item data entries included in each substitutable group category in the clinical usage hierarchy.

11. The system of claim 9, wherein the universal item database is a Global Unique Device Identification Database (GUDID).

12. The system of claim 9, wherein
at least one of the substitutable group categories in the clinical usage hierarchy is a graded substitutable group category, and
the one or more item entries and subset of item data entries included in the graded substitutable group category are organized into two or more graded tiers.

13. The system of claim 12, wherein the instructions when executed by the one or more processors further cause the one or more processors to:
deploy a third machine learning model on the one or more item entries and subset of item data entries included in the graded substitutable group category to organize the one or more item entries and subset of item data entries into the two or more graded tiers.

14. The system of claim 13, wherein the instructions when executed by the one or more processors further cause the one or more processors to:
receive a tier assignment to one of the two or more graded tiers for each of at least one of the item data entries in the subset of item data entries included in the graded substitutable group category; and
train the third machine learning model on the at least one of the item data entries in the subset of item data entries included in the graded substitutable group category based on the received tier assignment.

15. The system of claim 9, wherein the instructions when executed by the one or more processors further cause the one or more processors to:
transmit a user interface to a user device in communication with the one or more processors, that displays a plurality of form elements for entry of procedure data for a medical procedure associated with the practice area, wherein
the plurality of form elements includes at least one form element for selection of an item associated with at least one of the substitutable group categories in the clinical usage hierarchy, and
the user interface pre-populates one or more data values for the at least one form element based on the generated product formulary.

16. The system of claim 9, further comprising:
the one or more processors includes an artificial intelligence engine, wherein
the artificial intelligence engine is configured to deploy the first trained machine learning model.
